(19) United States
(12) Patent Application Publication
(10) Pub. No.: US 2024/0050421 A1
(43) Pub. Date: Feb. 15, 2024

(54) PHARMACEUTICAL FORMULATIONS OF A PHENOLIC TRPV1 AGONIST PRODRUG

(71) Applicant: Concentric Analgesics, Inc., San Francisco, CA (US)

(72) Inventors: Susan WOLLOWITZ, San Francisco, CA (US); John F. DONOVAN, San Francisco, CA (US)

(21) Appl. No.: 18/257,273

(22) PCT Filed: Dec. 13, 2021
(86) PCT No.: PCT/US2021/063121

§ 371 Date: Jun. 13, 2023

Related U.S. Application Data
(60) Provisional application No. 63/125,316, filed on Dec. 14, 2020.

(51) Int. Cl. A61K 31/445; A61K 9/19; A61K 47/12; A61K 47/26; A61J 1/14; A61M 5/19; A61K 45/06

(52) U.S. Cl. CPC A61K 31/445; A61K 9/19; A61K 47/12; A61K 47/26; A61J 1/1412; A61M 5/19; A61K 45/06

(57) ABSTRACT

Provided herein are pharmaceutical formulations comprising a transient receptor potential vanilloid 1 receptor (TRPV1) agonist, or a pharmaceutically acceptable salt thereof.

### CROSS-REFERENCE

This application claims benefit of U.S. Provisional Patent Application No. 63/125,316, filed on Dec. 14, 2020 which is incorporated herein by reference in its entirety.

### BACKGROUND OF THE INVENTION

More than 80% of patients who undergo surgical procedures experience acute postoperative pain and approximately 75% of those with postoperative pain report the severity as moderate, severe, or extreme (Apfelbaum et al., 2003; Gan et al., 2014). Evidence suggests that less than half of patients who undergo surgery report adequate postoperative pain relief (Apfelbaum et al., 2003). Inadequately controlled pain negatively affects quality of life, function, and functional recovery, the risk of post-surgical complications, and the risk of persistent postsurgical pain (Kehlet et al., 2006). Thus, there exists a need for medicaments with improved efficacy and longer duration of action for the treatment of post-surgical pain.

### SUMMARY OF THE INVENTION

In one aspect, described herein is a solid pharmaceutical composition comprising:
(a) about 0.25 mg to about 125 mg of (E)-2-methoxy-4-((8-methylnon-6-enamido)methyl)phenyl 2-((methylamino)methyl)piperidine-1-carboxylate, or a pharmaceutically acceptable salt thereof, and
(b) about 0.25 mg to about 75 mg of a buffering agent.

In some embodiments, the solid pharmaceutical composition comprises about 1 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 3 mg to about 75 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises (E)-2-methoxy-4-((8-methylnon-6-enamido)methyl)phenyl 2-((methylamino)methyl)piperidine-1-carboxylate hydrochloride salt.

In some embodiments, the solid pharmaceutical composition comprises about 0.5 mg to about 45 mg of a buffering agent. In some embodiments, the solid pharmaceutical composition comprises about 2 mg to about 35 mg of a buffering agent. In some embodiments, the buffering agent is selected from citrate, acetate, formate, glycine, maleate, tartrate, fumarate, and succinate. In some embodiments, the buffering agent is a citrate buffer. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.2 to about 10. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 3. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.5.

In some embodiments, the solid pharmaceutical composition further comprises a bulking agent. In some embodiments, the solid pharmaceutical composition further comprises about 0.1 mg to about 200 mg of a bulking agent. In some embodiments, the solid pharmaceutical composition further comprises about 1.0 mg to about 150 mg of a bulking agent. In some embodiments, the solid pharmaceutical composition further comprises about 10 mg to about 125 mg of a bulking agent. In some embodiments, the bulking agent is selected from mannitol, lactose, and polyvinylpyrrolidone. In some embodiments, the bulking agent is mannitol. In some embodiments, the bulking agent is lactose. In some embodiments, the solid pharmaceutical composition is provided in a sterile container. In some embodiments, the solid pharmaceutical composition is provided in a sterile container that allows for initial dilution within the container and optionally subsequent further dilution within or outside the container. In some embodiments, the sterile container comprises a vial with stopper. In some embodiments, the sterile container comprises a syringe cartridge. In some embodiments, the sterile container comprises a syringe cartridge comprising a two component syringe that also contains a diluent. In some embodiments, the sterile container comprises a kit or assembly of separate components packaged for a single use comprising one or more of the following components: diluent, container to hold a reconstituted solution of the solid pharmaceutical composition dissolved in the diluent, syringes, or devices to connect the container to other containers for transfer of the solid pharmaceutical composition, the diluent, or the reconstituted solution.

In another aspect, described herein is a liquid pharmaceutical formulation comprising (E)-2-methoxy-4-((8-methylnon-6-enamido)methyl)phenyl 2-((methylamino)methyl)piperidine-1-carboxylate, or a pharmaceutically acceptable salt thereof, wherein the concentration of (E)-2-methoxy-4-((8-methylnon-6-enamido)methyl)phenyl 2-((methylamino)methyl)piperidine-1-carboxylate, free base, is about 0.02 to about 2 mg/mL, the pH is 3.0 to 6.0, and the osmolarity is about 300 mOsm/kg to about 600 mOsm/kg. In some embodiments, the pH is 3.4 to 5.0. In some embodiments, the concentration of (E)-2-methoxy-4-((8-methylnon-6-enamido)methyl)phenyl 2-((methylamino)methyl)piperidine-1-carboxylate, free base, is about 0.05 mg/mL to about 1 mg/mL. In some embodiments, the concentration of (E)-2-methoxy-4-((8-methylnon-6-enamido)methyl)phenyl 2-((methylamino)methyl)piperidine-1-carboxylate, free base, is about 0.1 mg/mL to about 0.75 mg/mL. In some embodiments, the liquid pharmaceutical formulation comprises (E)-2-methoxy-4-((8-methylnon-6-enamido)methyl)phenyl 2-((methylamino)methyl)piperidine-1-carboxylate hydrochloride salt. In some embodiments, the liquid pharmaceutical formulation further comprises a buffering agent. In some embodiments, the buffering agent is selected from citrate, acetate, formate, glycine, maleate, tartrate, fumarate, and succinate. In some embodiments, the buffering agent is a citrate buffer. In some embodiments, the buffer concentration is about 0.05 mM to about 10 mM. In some embodiments, the buffer concentration is about 0.1 mM to about 5 mM. In some embodiments, the buffer concentration is about 0.2 mM to about 2 mM. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.25 to about 10. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 3. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.5. In some embodiments, the liquid pharmaceutical formulation further comprises a bulking agent. In some embodiments, the liquid pharmaceutical formulation further comprises about 0.005 to 0.4 weight percent of a bulking agent. In some embodiments, the liquid pharmaceutical formulation further comprises about 0.01 to 0.1 weight percent of a bulking agent. In some embodiments, the bulking agent is selected from mannitol, lactose, and polyvinylpyrrolidone. In some embodiments, the bulking agent is mannitol. In some embodiments, the bulking agent is lactose.

In another aspect is a liquid pharmaceutical formulation comprising:
a) about 0.05 mg/mL to about 0.75 mg/mL of (E)-2-methoxy-4-((8-methylnon-6-enamido)methyl)phenyl 2-((methylamino)methyl)piperidine-1-carboxylate, or a pharmaceutically acceptable salt thereof,
b) about 0.1 to 1.0 mM of a buffering agent;
c) pH of 3.0 to 6.0; and
d) osmolality of about 300 mOsm/kg to about 600 mOsm/kg.

In some embodiments, the liquid pharmaceutical formulation comprises about 1 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 3 mg to about 75 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.05 mg/mL to about 0.6 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.1 mg/mL to about 0.5 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation further comprises a local anesthetic. In some embodiments, the liquid pharmaceutical formulation further comprises a local anesthetic, wherein the local anesthetic is selected from bupivacaine, levobupivacaine, tetracaine, and ropivacaine.

In another aspect is a method of preparing a liquid pharmaceutical formulation, comprising dissolving a solid pharmaceutical composition described herein in a diluent.

In another aspect is a method of preparing a liquid pharmaceutical formulation, comprising dissolving a solid pharmaceutical composition described herein in a first portion of a diluent in a first container, and then transferring to a second container for dilution with a second portion of a diluent to a final volume and concentration. In some embodiments, the first container is attached in a sterile manner to the second container by a connection device and the first portion of diluent is used to flush the liquid pharmaceutical formulation into the second container.

In another aspect is a method of preparing a liquid pharmaceutical formulation, comprising a two compartment syringe containing a solid pharmaceutical composition described herein in a first compartment and a diluent in a second compartment, wherein the solid pharmaceutical composition in the first compartment and the diluent in the second compartment are combined to allow dissolution of the solid pharmaceutical composition in the diluent directly in the syringe.

In some embodiments, the diluent is a sterile solution. In some embodiments, the diluent is a saline solution. In some embodiments, the diluent is a Lactated Ringer's solution.

In another aspect is a method of treating or preventing pain in a subject in need thereof, comprising parenterally administering to the subject a liquid pharmaceutical formulation described herein. In some embodiments, the pain is post-surgical pain, post amputation pain, chronic post-surgical pain, and pain associated with acute traumatic injury. In some embodiments, the pain is postsurgical pain. In some embodiments, the postsurgical pain is pain from a laparotomy, hernia repair, thoracotomy, thoraco-abdominal incision, flank incision, total hip replacement, total knee replacement, ACL reconstruction, rotator cuff repair, bunionectomy, laparoscopy, dental extraction, or open reduction internal fixation of fractures. In some embodiments, the pain is pain associated with acute traumatic injury. In some embodiments, the pain associated with acute traumatic injury is pain from a long bone, short bone, flat bone, or irregular bone fracture. In some embodiments, the pain associated with acute traumatic injury pain is pain from a hip or rib fracture. In some embodiments, the pain is chronic post-surgical pain. In some embodiments, the chronic post-surgical pain is pain after mastectomy or lumpectomy. In some embodiments, the chronic post-surgical pain is pain after thoracotomy. In some embodiments, the chronic post-surgical pain is pain after amputation. In some embodiments, the chronic post-surgical pain is pain after hernia repair. In some embodiments, the pain is chronic pain. In some embodiments, the chronic pain is chronic pain associated with osteoarthritis. In some embodiments, the chronic pain is chronic pain associated with osteoarthritis of the knee. In some embodiments, the chronic pain is chronic musculoskeletal pain. In some embodiments, the chronic pain is chronic musculoskeletal pain of the lower back.

In some embodiments, 10 mL to 150 mL of the liquid pharmaceutical formulation is administered to the patient.

In another aspect is a solid pharmaceutical formulation comprising:
a) about 3-75 mg of (E)-2-methoxy-4-((8-methylnon-6-enamido)methyl)phenyl 2-((methylamino)methyl)piperidine-1-carboxylate hydrochloride salt;
b) about 20-150 micromoles of citrate buffer; and
c) about 10-100 mg of a bulking agent.

In another aspect is a liquid pharmaceutical formulation comprising:
a) about 0.15 to 0.75 mg/mL of (E)-2-methoxy-4-((8-methylnon-6-enamido)methyl)phenyl 2-((methylamino)methyl)piperidine-1-carboxylate hydrochloride salt;
b) about 0.25 to 1.25 mM of a citrate buffer at pH 3.4 to 5.0; and
c) osmolality of about 300 mOsm/kg to about 600 mOsm/kg.

### DETAILED DESCRIPTION

### Certain Terminology

Unless defined otherwise, all technical and scientific terms used herein have the same meaning as is commonly understood to which the claimed subject matter belongs. In the event that there are a plurality of definitions for terms herein, those in this section prevail. All patents, patent applications, publications, and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The term “about” when referring to a number or a numerical range means that the number or numerical range referred to is an approximation within experimental variability (or within statistical experimental error), and thus the number or numerical range varies between 1% and 15% of the stated number or numerical range.

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, and compositions described herein.

The terms “kit” and “article of manufacture” are used as synonyms.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, and swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition (e.g., arresting the development of the disease or condition), relieving the disease or condition, causing regression of the disease or condition, relieving a condition caused by the disease or condition, or stopping the symptoms of the disease or condition either prophylactically and/or therapeutically.

As used herein, amelioration of the symptoms of a particular disease, disorder, or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “acceptable” with respect to a formulation, composition or ingredient, as used herein, means having no persistent detrimental effect on the general health of the subject being treated.

By “pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical composition” refers to a mixture of a compound described herein with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the pharmaceutical composition that includes a compound described herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

“Bioavailability” refers to the percentage of the weight of the compound disclosed herein that is delivered into the general circulation of the animal or human being studied. The total exposure (AUC(0-∞)) of a drug when administered intravenously is usually defined as 100% bioavailable (F %). “Oral bioavailability” refers to the extent to which a compound disclosed herein, is absorbed into the general circulation when the pharmaceutical composition is taken orally as compared to intravenous injection.

Pain management in patients after surgery remains insufficient (Pogatzki-Zahn et al., 2012), and there is no ideal way to provide continuous, effective pain relief beyond 12-18 hours after surgery. Systemic pharmacological therapies remain the mainstay of postoperative pain relief, with opioids a key component, especially for moderate-to-severe pain. Systemic opioids are effective, but increase cost and morbidity, especially due to known safety issues such as respiratory depression, gastrointestinal dysfunction, and abuse. Non-opioid analgesics including acetaminophen, nonselective NSAIDs, and selective COX-2 inhibitors are useful for the treatment of light-to-moderate pain and are part of a balanced multimodal pain treatment (Pogatzki-Zahn et al., 2012). These products also have known safety risks. The use of peripheral regional anesthetic techniques have been shown to be effective as a component of multimodal analgesia for management of postoperative pain associated with a number of surgical procedures, including thoracotomy, lower extremity joint surgery, shoulder surgery, cesarean section, hemorrhoid surgery, and circumcision. It is recommended that clinicians should consider use of surgical site-specific or peripheral regional analgesic techniques in adults and children as part of multimodal analgesia, particularly in patients who undergo lower extremity and upper extremity surgical procedures (Chou et al., 2016).

Capsaicin (8-methyl-N-vanillyl-6-nonenamide) is a highly selective agonist for transient receptor potential vanilloid 1 receptor (TRPV1; formerly known as vanilloid receptor 1 (VR1)), a ligand-gated, non-selective cation channel. TRPV1 is preferentially expressed on small-diameter sensory neurons, predominately on C-fibers and to a lesser extent A-delta fibers which specialize in the detection of painful or noxious sensations. TRPV1 responds to stimuli including capsaicin, heat, and extracellular acidification, and will integrate simultaneous exposures to these stimuli. (Caterina M J, Julius D. The vanilloid receptor: a molecular gateway to the pain pathway. Annu Rev Neurosci. 2001. 24:487-517).

TRPV1 agonists, such as capsaicin, have been shown to diminish pain in various settings. (E)-2-methoxy-4-((8-methylnon-6-enamido)methyl)phenyl 2-((methylamino)methyl)piperidine-1-carboxylate is a capsaicin prodrug.

The chemical structure of (E)-2-methoxy-4-((8-methylnon-6-enamido)methyl)phenyl 2-((methylamino)methyl)piperidine-1-carboxylate hydrochloride is shown below:

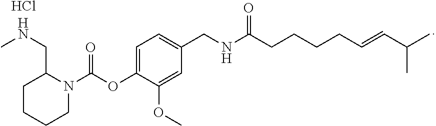 [SMILES: Cl ; CNCC1CCCCN1C(=O)Oc1ccc(CNC(=O)CCCC/C=C/C(C)C)cc1OC]

In some embodiments, (E)-2-methoxy-4-((8-methylnon-6-enamido)methyl)phenyl 2-((methylamino)methyl)piperidine-1-carboxylate is in the free base form. In some embodiments, (E)-2-methoxy-4-((8-methylnon-6-enamido)methyl)phenyl 2-((methylamino)methyl)piperidine-1-carboxylate is in a pharmaceutically acceptable salt form. In some embodiments, (E)-2-methoxy-4-((8-methylnon-6-enamido)methyl)phenyl 2-((methylamino)methyl)piperidine-1-carboxylate is a hydrochloride salt.

(E)-2-methoxy-4-((8-methylnon-6-enamido)methyl)phenyl 2-((methylamino)methyl)piperidine-1-carboxylate (Compound 1) releases capsaicin and cyclic urea Compound 2 (2-methylhexahydroimidazo[1,5-a]pyridin-3(2H)-one) under well-defined rates via a pH driven, intra-molecular cyclization release reaction after Compound 1 has been delivered to the body and/or is exposed to specific physiological conditions:

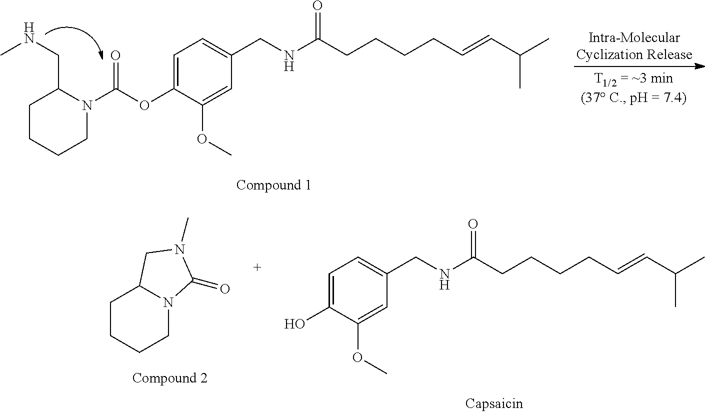 [SMILES: CNCC1CCCCN1C(=O)Oc1ccc(CNC(=O)CCCC/C=C/C(C)C)cc1OC ; CN1CC2CCCCN2C1=O ; COc1cc(CNC(=O)CCCC/C=C/C(C)C)ccc1O]

Compound 1 has significantly higher hydrophilicity/water solubility than capsaicin and, hence, is better able to be incorporated into commonly used aqueous formulations. The improved water solubility of Compound 1 is significant when co-administering other medications, especially when administering multiple sterile agents via injection.

In some embodiments, Compound 1 eliminates the reliance on special requirements for formulations or delivery devices for capsaicin in order to 1) accommodate the very low water solubility of capsaicin and 2) reduce the acute pungency associated with the administration of capsaicin.

In some embodiments, the rate at which Compound 1 releases capsaicin is modified by the addition of buffers. In some embodiments, the addition of a buffer provides a time window where conversion to capsaicin in the pharmaceutical preparation is significantly delayed until parenteral administration occurs.

### Solid Pharmaceutical Compositions

In some embodiments described herein is a solid pharmaceutical composition comprising:
(a) about 0.25 mg to about 125 mg of (E)-2-methoxy-4-((8-methylnon-6-enamido)methyl)phenyl 2-((methylamino)methyl)piperidine-1-carboxylate, or a pharmaceutically acceptable salt thereof, and
(b) about 0.25 mg to about 75 mg of a buffering agent.

In some embodiments, the solid pharmaceutical composition comprises about 0.5 mg to about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 80 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 70 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 55 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 45 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 3 mg to about 75 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 3 mg to about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 5 mg to about 55 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 5 mg to about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 5 mg to about 45 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 5 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 5 mg to about 125 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 10 mg to about 125 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 10 mg to about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 15 mg to about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 15 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 15 mg to about 80 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 15 mg to about 70 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 15 mg to about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 20 mg to about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 20 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 25 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 30 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 125 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 110 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 80 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 70 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 55 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 45 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 35 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 30 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 25 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 20 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 15 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 10 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 5 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 1 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 0.5 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises about 0.25 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the solid pharmaceutical composition comprises (E)-2-methoxy-4-((8-methylnon-6-enamido)methyl)phenyl 2-((methylamino)methyl)piperidine-1-carboxylate hydrochloride salt.

In some embodiments, the solid pharmaceutical composition comprises about 0.5 mg to about 45 mg of a buffering agent. In some embodiments, the solid pharmaceutical composition comprises about 1 mg to about 40 mg of a buffering agent. In some embodiments, the solid pharmaceutical composition comprises about 2 mg to about 35 mg of a buffering agent. In some embodiments, the solid pharmaceutical composition comprises about 2 mg to about 30 mg of a buffering agent. In some embodiments, the solid pharmaceutical composition comprises about 2 mg to about 25 mg of a buffering agent. In some embodiments, the solid pharmaceutical composition comprises about 2 mg to about 20 mg of a buffering agent. In some embodiments, the solid pharmaceutical composition comprises about 2 mg to about 15 mg of a buffering agent. In some embodiments, the solid pharmaceutical composition comprises about 2 mg to about 10 mg of a buffering agent. In some embodiments, the solid pharmaceutical composition comprises about 5 mg to about 25 mg of a buffering agent. In some embodiments, the solid pharmaceutical composition comprises about 5 mg to about 20 mg of a buffering agent. In some embodiments, the solid pharmaceutical composition comprises about 5 mg to about 15 mg of a buffering agent. In some embodiments, the solid pharmaceutical composition comprises about 45 mg of a buffering agent. In some embodiments, the solid pharmaceutical composition comprises about 40 mg of a buffering agent. In some embodiments, the solid pharmaceutical composition comprises about 35 mg of a buffering agent. In some embodiments, the solid pharmaceutical composition comprises about 30 mg of a buffering agent. In some embodiments, the solid pharmaceutical composition comprises about 25 mg of a buffering agent. In some embodiments, the solid pharmaceutical composition comprises about 20 mg of a buffering agent. In some embodiments, the solid pharmaceutical composition comprises about 19 mg of a buffering agent. In some embodiments, the solid pharmaceutical composition comprises about 18 mg of a buffering agent. In some embodiments, the solid pharmaceutical composition comprises about 17 mg of a buffering agent. In some embodiments, the solid pharmaceutical composition comprises about 16 mg of a buffering agent. In some embodiments, the solid pharmaceutical composition comprises about 15 mg of a buffering agent. In some embodiments, the solid pharmaceutical composition comprises about 14 mg of a buffering agent. In some embodiments, the solid pharmaceutical composition comprises about 13 mg of a buffering agent. In some embodiments, the solid pharmaceutical composition comprises about 12 mg of a buffering agent. In some embodiments, the solid pharmaceutical composition comprises about 11 mg of a buffering agent. In some embodiments, the solid pharmaceutical composition comprises about 10 mg of a buffering agent. In some embodiments, the solid pharmaceutical composition comprises about 9 mg of a buffering agent. In some embodiments, the solid pharmaceutical composition comprises about 8 mg of a buffering agent. In some embodiments, the solid pharmaceutical composition comprises about 7 mg of a buffering agent. In some embodiments, the solid pharmaceutical composition comprises about 6 mg of a buffering agent. In some embodiments, the solid pharmaceutical composition comprises about 5 mg of a buffering agent. In some embodiments, the solid pharmaceutical composition comprises about 3 mg of a buffering agent. In some embodiments, the solid pharmaceutical composition comprises about 1 mg of a buffering agent. In some embodiments, the solid pharmaceutical composition comprises about 0.5 mg of a buffering agent. In some embodiments, the buffering agent is selected from citrate, acetate, formate, glycine, maleate, tartrate, fumarate, and succinate. In some embodiments, the buffering agent is a citrate buffer. In some embodiments, the citrate buffer comprises citric acid monohydrate and sodium citrate dihydrate. In some embodiments, the buffering agent is an acetate buffer. In some embodiments, the buffering agent is a glycine buffer. In some embodiments, the buffering agent is an acetate buffer. In some embodiments, the buffering agent is a maleate buffer. In some embodiments, the buffering agent is a tartrate buffer. In some embodiments, the buffering agent is a fumarate buffer. In some embodiments, the buffering agent is a succinate buffer.

In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.2 to about 10. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.2 to about 5. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 5. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 3. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.5. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.

In some embodiments, the solid pharmaceutical composition further comprises a bulking agent. In some embodiments, the solid pharmaceutical composition further comprises about 0.1 mg to about 200 mg of a bulking agent. In some embodiments, the solid pharmaceutical composition further comprises about 0.5 mg to about 175 mg of a bulking agent. In some embodiments, the solid pharmaceutical composition further comprises about 1.0 mg to about 150 mg of a bulking agent. In some embodiments, the solid pharmaceutical composition further comprises about 5 mg to about 150 mg of a bulking agent. In some embodiments, the solid pharmaceutical composition further comprises about 10 mg to about 125 mg of a bulking agent. In some embodiments, the solid pharmaceutical composition further comprises about 10 mg to about 100 mg of a bulking agent. In some embodiments, the solid pharmaceutical composition further comprises about 10 mg to about 80 mg of a bulking agent. In some embodiments, the solid pharmaceutical composition further comprises about 20 mg to about 80 mg of a bulking agent. In some embodiments, the solid pharmaceutical composition further comprises about 20 mg to about 70 mg of a bulking agent. In some embodiments, the solid pharmaceutical composition further comprises about 200 mg of a bulking agent. In some embodiments, the solid pharmaceutical composition further comprises about 175 mg of a bulking agent. In some embodiments, the solid pharmaceutical composition further comprises about 150 mg of a bulking agent. In some embodiments, the solid pharmaceutical composition further comprises about 125 mg of a bulking agent. In some embodiments, the solid pharmaceutical composition further comprises about 100 mg of a bulking agent. In some embodiments, the solid pharmaceutical composition further comprises about 75 mg of a bulking agent. In some embodiments, the solid pharmaceutical composition further comprises about 70 mg of a bulking agent. In some embodiments, the solid pharmaceutical composition further comprises about 65 mg of a bulking agent. In some embodiments, the solid pharmaceutical composition further comprises about 60 mg of a bulking agent. In some embodiments, the solid pharmaceutical composition further comprises about 55 mg of a bulking agent. In some embodiments, the solid pharmaceutical composition further comprises about 50 mg of a bulking agent. In some embodiments, the solid pharmaceutical composition further comprises about 45 mg of a bulking agent. In some embodiments, the solid pharmaceutical composition further comprises about 40 mg of a bulking agent. In some embodiments, the solid pharmaceutical composition further comprises about 35 mg of a bulking agent. In some embodiments, the solid pharmaceutical composition further comprises about 30 mg of a bulking agent. In some embodiments, the solid pharmaceutical composition further comprises about 25 mg of a bulking agent. In some embodiments, the solid pharmaceutical composition further comprises about 20 mg of a bulking agent. In some embodiments, the solid pharmaceutical composition further comprises about 15 mg of a bulking agent. In some embodiments, the solid pharmaceutical composition further comprises about 10 mg of a bulking agent. In some embodiments, the solid pharmaceutical composition further comprises about 5 mg of a bulking agent. In some embodiments, the solid pharmaceutical composition further comprises about 1 mg of a bulking agent. In some embodiments, the solid pharmaceutical composition further comprises about 0.5 mg of a bulking agent. In some embodiments, the solid pharmaceutical composition further comprises about 0.1 mg of a bulking agent. In some embodiments, the bulking agent is selected from mannitol, lactose, and polyvinylpyrrolidone. In some embodiments, the bulking agent is mannitol. In some embodiments, the bulking agent is lactose. In some embodiments, the bulking agent is polyvinylpyrrolidone.

In some embodiments, the solid pharmaceutical composition is provided in a sterile container. In some embodiments, the solid pharmaceutical composition is provided in a sterile container that allows for initial dilution within the container and optionally subsequent further dilution within or outside the container. In some embodiments, the sterile container comprises a vial with stopper. In some embodiments, the sterile container comprises a syringe cartridge. In some embodiments, the sterile container comprises a syringe cartridge comprising a two component syringe that also contains a diluent. In some embodiments, the sterile container comprises a kit or assembly of separate components packaged for a single use comprising one or more of the following components: diluent, container to hold a reconstituted solution of the solid pharmaceutical composition dissolved in the diluent, syringes, or devices to connect the container to other containers for transfer of the solid pharmaceutical composition, the diluent, or the reconstituted solution.

### Liquid Pharmaceutical Formulations

In some embodiments described herein is a liquid pharmaceutical formulation comprising (E)-2-methoxy-4-((8-methylnon-6-enamido)methyl)phenyl 2-((methylamino)methyl)piperidine-1-carboxylate, or a pharmaceutically acceptable salt thereof, wherein the concentration of (E)-2-methoxy-4-((8-methylnon-6-enamido)methyl)phenyl 2-((methylamino)methyl)piperidine-1-carboxylate, free base, is about 0.02 to about 2 mg/mL, the pH is 3.0 to 6.0, and the osmolarity is about 300 mOsm/kg to about 600 mOsm/kg.

In some embodiments, the pH is 3.0 to 5.5. In some embodiments, the pH is 3.2 to 5.5. In some embodiments, the pH is 3.2 to 5.0. In some embodiments, the pH is 3.4 to 5.0. In some embodiments, the pH is 3.6 to 5.0. In some embodiments, the pH is about 6.0. In some embodiments, the pH is about 5.7. In some embodiments, the pH is about 5.5. In some embodiments, the pH is about 5.2. In some embodiments, the pH is about 5.0. In some embodiments, the pH is about 4.8. In some embodiments, the pH is about 4.5. In some embodiments, the pH is about 4.2. In some embodiments, the pH is about 4.0. In some embodiments, the pH is about 3.8. In some embodiments, the pH is about 3.5. In some embodiments, the pH is about 3.4. In some embodiments, the pH is about 3.2. In some embodiments, the pH is about 3.0.

In some embodiments, the concentration of (E)-2-methoxy-4-((8-methylnon-6-enamido)methyl)phenyl 2-((methylamino)methyl)piperidine-1-carboxylate, free base, is about 0.05 mg/mL to about 1 mg/mL. In some embodiments, the concentration of (E)-2-methoxy-4-((8-methylnon-6-enamido)methyl)phenyl 2-((methylamino)methyl)piperidine-1-carboxylate, free base, is about 0.075 mg/mL to about 1 mg/mL. In some embodiments, the concentration of (E)-2-methoxy-4-((8-methylnon-6-enamido)methyl)phenyl 2-((methylamino)methyl)piperidine-1-carboxylate, free base, is about 0.1 mg/mL to about 1 mg/mL. In some embodiments, the concentration of (E)-2-methoxy-4-((8-methylnon-6-enamido)methyl)phenyl 2-((methylamino)methyl)piperidine-1-carboxylate, free base, is about 0.1 mg/mL to about 0.75 mg/mL. In some embodiments, the liquid pharmaceutical formulation comprises (E)-2-methoxy-4-((8-methylnon-6-enamido)methyl)phenyl 2-((methylamino)methyl)piperidine-1-carboxylate hydrochloride salt.

In some embodiments, the liquid pharmaceutical formulation further comprises a buffering agent. In some embodiments, the buffering agent is selected from citrate, acetate, formate, glycine, maleate, tartrate, fumarate, and succinate buffers. In some embodiments, the buffering agent is a citrate buffer. In some embodiments, the buffering agent is an acetate buffer. In some embodiments, the buffering agent is a glycine buffer. In some embodiments, the buffering agent is an acetate buffer. In some embodiments, the buffering agent is a maleate buffer. In some embodiments, the buffering agent is a tartrate buffer. In some embodiments, the buffering agent is a fumarate buffer. In some embodiments, the buffering agent is a succinate buffer. In some embodiments, the buffer concentration is about 0.05 mM to about 10 mM. In some embodiments, the buffer concentration is about 0.075 mM to about 10 mM. In some embodiments, the buffer concentration is about 0.1 mM to about 10 mM. In some embodiments, the buffer concentration is about 0.1 mM to about 7.5 mM. In some embodiments, the buffer concentration is about 0.1 mM to about 5 mM. In some embodiments, the buffer concentration is about 0.1 mM to about 1 mM. In some embodiments, the buffer concentration is about 0.2 mM to about 5 mM. In some embodiments, the buffer concentration is about 0.2 mM to about 2 mM. In some embodiments, the buffer concentration is about 10 mM. In some embodiments, the buffer concentration is about 7.5 mM. In some embodiments, the buffer concentration is about 5 mM. In some embodiments, the buffer concentration is about 2.5 mM. In some embodiments, the buffer concentration is about 2 mM. In some embodiments, the buffer concentration is about 1 mM. In some embodiments, the buffer concentration is about 0.875 mM. In some embodiments, the buffer concentration is about 0.75 mM. In some embodiments, the buffer concentration is about 0.625 mM. In some embodiments, the buffer concentration is about 0.5 mM. In some embodiments, the buffer concentration is about 0.25 mM. In some embodiments, the buffer concentration is about 0.1 mM. In some embodiments, the buffer concentration is about 0.05 mM. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.25 to about 10. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.25 to about 5. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.25 to about 3. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 3. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 2.5. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 2. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.5. In some embodiments, the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.0.

In some embodiments, the liquid pharmaceutical formulation further comprises a bulking agent. In some embodiments, the liquid pharmaceutical formulation further comprises about 0.005 to 0.4 weight percent of a bulking agent. In some embodiments, the liquid pharmaceutical formulation further comprises about 0.005 to 0.3 weight percent of a bulking agent. In some embodiments, the liquid pharmaceutical formulation further comprises about 0.005 to 0.2 weight percent of a bulking agent. In some embodiments, the liquid pharmaceutical formulation further comprises about 0.01 to 0.2 weight percent of a bulking agent. In some embodiments, the liquid pharmaceutical formulation further comprises about 0.01 to 0.1 weight percent of a bulking agent.

In some embodiments, the liquid pharmaceutical formulation further comprises a bulking agent. In some embodiments, the liquid pharmaceutical formulation further comprises about 0.001 mg/mL to about 2 mg/mL of a bulking agent. In some embodiments, the liquid pharmaceutical formulation further comprises about 0.005 mg/mL to about 2 mg/mL of a bulking agent. In some embodiments, the liquid pharmaceutical formulation further comprises about 0.01 mg/mL to about 2 mg/mL of a bulking agent. In some embodiments, the liquid pharmaceutical formulation further comprises about 0.05 mg/mL to about 2 mg/mL of a bulking agent. In some embodiments, the liquid pharmaceutical formulation further comprises about 0.05 mg/mL to about 1.5 mg/mL of a bulking agent. In some embodiments, the liquid pharmaceutical formulation further comprises about 0.1 mg/mL to about 1.5 mg/mL of a bulking agent. In some embodiments, the liquid pharmaceutical formulation further comprises about 0.1 mg/mL to about 1 mg/mL of a bulking agent. In some embodiments, the liquid pharmaceutical formulation further comprises about 0.2 mg/mL to about 1 mg/mL of a bulking agent. In some embodiments, the liquid pharmaceutical formulation further comprises about 0.2 mg/mL to about 0.8 mg/mL of a bulking agent. In some embodiments, the liquid pharmaceutical formulation further comprises about 2 mg/mL of a bulking agent. In some embodiments, the liquid pharmaceutical formulation further comprises about 1.5 mg/mL of a bulking agent. In some embodiments, the liquid pharmaceutical formulation further comprises about 1.25 mg/mL of a bulking agent. In some embodiments, the liquid pharmaceutical formulation further comprises about 1 mg/mL of a bulking agent. In some embodiments, the liquid pharmaceutical formulation further comprises about 0.9 mg/mL of a bulking agent. In some embodiments, the liquid pharmaceutical formulation further comprises about 0.8 mg/mL of a bulking agent. In some embodiments, the liquid pharmaceutical formulation further comprises about 0.7 mg/mL of a bulking agent. In some embodiments, the liquid pharmaceutical formulation further comprises about 0.6 mg/mL of a bulking agent. In some embodiments, the liquid pharmaceutical formulation further comprises about 0.5 mg/mL of a bulking agent. In some embodiments, the liquid pharmaceutical formulation further comprises about 0.4 mg/mL of a bulking agent. In some embodiments, the liquid pharmaceutical formulation further comprises about 0.3 mg/mL of a bulking agent. In some embodiments, the liquid pharmaceutical formulation further comprises about 0.3 mg/mL of a bulking agent. In some embodiments, the liquid pharmaceutical formulation further comprises about 0.2 mg/mL of a bulking agent. In some embodiments, the liquid pharmaceutical formulation further comprises about 0.1 mg/mL of a bulking agent. In some embodiments, the liquid pharmaceutical formulation further comprises about 0.05 mg/mL of a bulking agent. In some embodiments, the liquid pharmaceutical formulation further comprises about 0.01 mg/mL of a bulking agent. In some embodiments, the liquid pharmaceutical formulation further comprises about 0.005 mg/mL of a bulking agent. In some embodiments, the liquid pharmaceutical formulation further comprises about 0.001 mg/mL of a bulking agent.

In some embodiments, the bulking agent is selected from mannitol, lactose, and polyvinylpyrrolidone. In some embodiments, the bulking agent is mannitol. In some embodiments, the bulking agent is lactose. In some embodiments, the bulking agent is polyvinylpyrrolidone.

In another aspect is a liquid pharmaceutical formulation comprising:
a) about 0.05 mg/mL to about 0.75 mg/mL of (E)-2-methoxy-4-((8-methylnon-6-enamido)methyl)phenyl 2-((methylamino)methyl)piperidine-1-carboxylate, or a pharmaceutically acceptable salt thereof,
b) about 0.1 to 1.0 mM of a buffering agent; and,
c) pH of 3.0 to 6.0; and
(d) has an osmolality of about 300 mOsm/kg to about 600 mOsm/kg.

In some embodiments, the liquid pharmaceutical formulation comprises about 0.01 mg/mL to about 1 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.01 mg/mL to about 0.9 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.01 mg/mL to about 0.8 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.01 mg/mL to about 0.7 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.05 mg/mL to about 0.7 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.05 mg/mL to about 0.6 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.1 mg/mL to about 0.7 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.1 mg/mL to about 0.6 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.1 mg/mL to about 0.5 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.8 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.75 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.7 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.65 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.6 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.55 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.5 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.45 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.4 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.35 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.3 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.25 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.2 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.15 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.1 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.05 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.01 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.005 mg/mL of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.001 mg/mL of (E)-2-methoxy-4-((8-methylnon-6-enamido)methyl)phenyl 2-((methylamino)methyl)piperidine-1-carboxylate, or a pharmaceutically acceptable salt thereof.

In some embodiments, the liquid pharmaceutical formulation comprises about 0.5 mg to about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 80 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 70 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 55 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 45 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 3 mg to about 75 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 3 mg to about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 5 mg to about 55 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 5 mg to about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 5 mg to about 45 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 5 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 5 mg to about 125 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 10 mg to about 125 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 10 mg to about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 15 mg to about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 15 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 15 mg to about 80 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 15 mg to about 70 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 15 mg to about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 20 mg to about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 20 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 25 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 30 mg to about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 125 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 110 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 100 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 90 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 80 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 70 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 60 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 55 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 50 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 45 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 40 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 35 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 30 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 25 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 20 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 15 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 10 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 5 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 1 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.5 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises about 0.25 mg of (E)-2-methoxy-4-((8-methylnon-6-enamido)methyl)phenyl 2-((methylamino)methyl)piperidine-1-carboxylate, or a pharmaceutically acceptable salt thereof. In some embodiments, the liquid pharmaceutical formulation comprises (E)-2-methoxy-4-((8-methylnon-6-enamido)methyl)phenyl 2-((methylamino)methyl)piperidine-1-carboxylate hydrochloride salt.

In some embodiments, the liquid pharmaceutical formulation further comprises a local anesthetic. In some embodiments, the liquid pharmaceutical formulation further comprises a local anesthetic, wherein the local anesthetic is selected from bupivacaine, levobupivacaine, tetracaine, and ropivacaine. In some embodiments, the liquid pharmaceutical formulation further comprises a local anesthetic, wherein the local anesthetic is bupivacaine. In some embodiments, the liquid pharmaceutical formulation further comprises a local anesthetic, wherein the local anesthetic is levobupivacaine. In some embodiments, the liquid pharmaceutical formulation further comprises a local anesthetic, wherein the local anesthetic is tetracaine. In some embodiments, the liquid pharmaceutical formulation further comprises a local anesthetic, wherein the local anesthetic is ropivacaine.

In some embodiments is a method of preparing a liquid pharmaceutical formulation, comprising dissolving a solid pharmaceutical composition described herein in a diluent. In some embodiments is a method of preparing a liquid pharmaceutical formulation, comprising dissolving a solid pharmaceutical composition described herein in a first portion of a diluent in a first container, and then transferring to a second container for dilution with a second portion of a diluent to a final volume and concentration. In some embodiments, the first container is attached in a sterile manner to the second container by a connection device and the first portion of diluent is used to flush the liquid pharmaceutical formulation into the second container. In some embodiments is a method of preparing a liquid pharmaceutical formulation, comprising a two compartment syringe containing a solid pharmaceutical composition described herein in a first compartment and a diluent in a second compartment, wherein the solid pharmaceutical composition in the first compartment and the diluent in the second compartment are combined to allow dissolution of the solid pharmaceutical composition in the diluent directly in the syringe. In some embodiments, the diluent is a sterile solution. In some embodiments, the diluent is a saline solution. In some embodiments, the diluent is a Lactated Ringer's solution.

In some embodiments is a solid pharmaceutical formulation comprising:
a) about 3-75 mg of (E)-2-methoxy-4-((8-methylnon-6-enamido)methyl)phenyl 2-((methylamino)methyl)piperidine-1-carboxylate hydrochloride salt;
b) about 20-150 micromoles of citrate buffer; and
c) about 10-100 mg of a bulking agent.

In some embodiments is a solid pharmaceutical formulation comprising:
a) about 16 mg of (E)-2-methoxy-4-((8-methylnon-6-enamido)methyl)phenyl 2-((methylamino)methyl)piperidine-1-carboxylate hydrochloride salt;
b) about 5 mg citric acid monohydrate;
c) about 2 mg sodium citrate, dihydrate; and
d) about 25 mg of mannitol.

In some embodiments is a solid pharmaceutical formulation comprising:
a) about 39 mg of (E)-2-methoxy-4-((8-methylnon-6-enamido)methyl)phenyl 2-((methylamino)methyl)piperidine-1-carboxylate hydrochloride salt;
b) about 12 mg citric acid monohydrate;
c) about 5 mg sodium citrate, dihydrate; and
d) about 60 mg of mannitol.

In some embodiments is a solid pharmaceutical formulation comprising:
a) about 39 mg of (E)-2-methoxy-4-((8-methylnon-6-enamido)methyl)phenyl 2-((methylamino)methyl)piperidine-1-carboxylate hydrochloride salt;
b) about 10 mg citric acid monohydrate;
c) about 4 mg sodium citrate, dihydrate; and
d) about 48 mg of mannitol.

In some embodiments is a solid pharmaceutical formulation comprising:
a) about 65 mg of (E)-2-methoxy-4-((8-methylnon-6-enamido)methyl)phenyl 2-((methylamino)methyl)piperidine-1-carboxylate hydrochloride salt;
b) about 21 mg citric acid monohydrate;
c) about 8 mg sodium citrate, dihydrate; and
d) about 100 mg of mannitol.

In some embodiments is a liquid pharmaceutical formulation comprising:
a) about 0.15 to 0.75 mg/mL of (E)-2-methoxy-4-((8-methylnon-6-enamido)methyl)phenyl 2-((methylamino)methyl)piperidine-1-carboxylate hydrochloride salt;
b) about 0.25 to 1.25 mM of a citrate buffer at pH 3.4 to 5.0; and
c) has an osmolality of about 300 mOsm/kg to about 600 mOsm/kg.

In some embodiments is a liquid pharmaceutical formulation comprising:
a) about 0.15 to 0.75 mg/mL of (E)-2-methoxy-4-((8-methylnon-6-enamido)methyl)phenyl 2-((methylamino)methyl)piperidine-1-carboxylate hydrochloride salt;
b) about 0.25 to 1.25 mM of a citrate buffer at pH 3.4 to 5.0; and
c) about 0.005 to 0.4 weight percent of a bulking agent.

In some embodiments is a liquid pharmaceutical formulation comprising:
a) about 0.15 to 0.75 mg/mL of (E)-2-methoxy-4-((8-methylnon-6-enamido)methyl)phenyl 2-((methylamino)methyl)piperidine-1-carboxylate hydrochloride salt;
b) about 0.25 to 1.25 mM of a citrate buffer at pH 3.4 to 5.0;
c) about 0.005 to 0.4 weight percent of a bulking agent; and
d) osmolality of about 300 mOsm/kg to about 600 mOsm/kg.

In some embodiments is a liquid pharmaceutical formulation comprising:
a) about 0.125 mg/mL of (E)-2-methoxy-4-((8-methylnon-6-enamido)methyl)phenyl 2-((methylamino)methyl)piperidine-1-carboxylate free base;
b) about 0.26 mM of a citrate buffer at pH 3.4 to 5.0; and
c) about 0.2 mg/mL of mannitol.

In some embodiments is a liquid pharmaceutical formulation comprising:
a) about 0.125 mg/mL of (E)-2-methoxy-4-((8-methylnon-6-enamido)methyl)phenyl 2-((methylamino)methyl)piperidine-1-carboxylate free base;
b) about 0.26 mM of a citrate buffer at pH 3.4 to 5.0;
c) about 0.2 mg/mL of mannitol; and
d) osmolality of about 300 mOsm/kg to about 600 mOsm/kg.

In some embodiments is a liquid pharmaceutical formulation comprising:
a) about 0.3 mg/mL of (E)-2-methoxy-4-((8-methylnon-6-enamido)methyl)phenyl 2-((methylamino)methyl)piperidine-1-carboxylate free base;
b) about 0.63 mM of a citrate buffer at pH 3.4 to 5.0; and
c) about 0.5 mg/mL of mannitol.

In some embodiments is a liquid pharmaceutical formulation comprising:
a) about 0.3 mg/mL of (E)-2-methoxy-4-((8-methylnon-6-enamido)methyl)phenyl 2-((methylamino)methyl)piperidine-1-carboxylate free base;
b) about 0.63 mM of a citrate buffer at pH 3.4 to 5.0;
c) about 0.5 mg/mL of mannitol; and
d) osmolality of about 300 mOsm/kg to about 600 mOsm/kg.

In some embodiments is a liquid pharmaceutical formulation comprising:
a) about 0.3 mg/mL of (E)-2-methoxy-4-((8-methylnon-6-enamido)methyl)phenyl 2-((methylamino)methyl)piperidine-1-carboxylate free base;
b) about 0.5 mM of a citrate buffer at pH 3.4 to 5.0; and
c) about 0.4 mg/mL of mannitol.

In some embodiments is a liquid pharmaceutical formulation comprising:
a) about 0.3 mg/mL of (E)-2-methoxy-4-((8-methylnon-6-enamido)methyl)phenyl 2-((methylamino)methyl)piperidine-1-carboxylate free base;
b) about 0.5 mM of a citrate buffer at pH 3.4 to 5.0;
c) about 0.4 mg/mL of mannitol; and
d) osmolality of about 300 mOsm/kg to about 600 mOsm/kg.

In some embodiments is a liquid pharmaceutical formulation comprising:
a) about 0.5 mg/mL of (E)-2-methoxy-4-((8-methylnon-6-enamido)methyl)phenyl 2-((methylamino)methyl)piperidine-1-carboxylate free base;
b) about 1.05 mM of a citrate buffer at pH 3.4 to 5.0; and
c) about 0.8 mg/mL of mannitol.

In some embodiments is a liquid pharmaceutical formulation comprising:
a) about 0.5 mg/mL of (E)-2-methoxy-4-((8-methylnon-6-enamido)methyl)phenyl 2-((methylamino)methyl)piperidine-1-carboxylate free base;
b) about 1.05 mM of a citrate buffer at pH 3.4 to 5.0;
c) about 0.8 mg/mL of mannitol; and
d) osmolality of about 300 mOsm/kg to about 600 mOsm/kg.

### Methods of Treatment

In some embodiments is a method of treating or preventing pain in a subject in need thereof, comprising parenterally administering to the subject a liquid pharmaceutical formulation described herein. In some embodiments, the pain is post-surgical pain, post amputation pain, chronic post-surgical pain, and pain associated with acute traumatic injury. In some embodiments, the pain is post-surgical pain. In some embodiments, the post-surgical pain is pain from a laparotomy, hernia repair, thoracotomy, thoraco-abdominal incision, flank incision, total hip replacement, total knee replacement, ACL reconstruction, rotator cuff repair, bunionectomy, laparoscopy, dental extraction, or open reduction internal fixation of fractures. In some embodiments, the post-surgical pain is pain from a laparotomy. In some embodiments, the post-surgical pain is pain from hernia repair. In some embodiments, the post-surgical pain is pain from ventral hernia repair. In some embodiments, the post-surgical pain is pain from a thoracotomy. In some embodiments, the post-surgical pain is pain from a thoraco-abdominal incision. In some embodiments, the post-surgical pain is pain from a flank incision. In some embodiments, the post-surgical pain is pain from a total hip replacement. In some embodiments, the post-surgical pain is pain from a total knee replacement. In some embodiments, the post-surgical pain is pain from an ACL reconstruction. In some embodiments, the post-surgical pain is pain from a rotator cuff repair. In some embodiments, the post-surgical pain is pain from a bunionectomy. In some embodiments, the post-surgical pain is pain from a laparoscopy. In some embodiments, the post-surgical pain is pain from a dental extraction. In some embodiments, the post-surgical pain is pain from an open reduction internal fixation of fractures. In some embodiments, the pain is pain associated with acute traumatic injury. In some embodiments, the pain associated with acute traumatic injury is pain from a long bone, short bone, flat bone, or irregular bone fracture. In some embodiments, the pain associated with acute traumatic injury pain is pain from a hip or rib fracture. In some embodiments, the pain is chronic post-surgical pain. In some embodiments, the chronic post-surgical pain is pain after mastectomy or lumpectomy. In some embodiments, the chronic post-surgical pain is pain after thoracotomy. In some embodiments, the chronic post-surgical pain is pain after amputation. In some embodiments, the chronic post-surgical pain is post amputation pain. In some embodiments, the chronic post-surgical pain is pain after hernia repair. In some embodiments, the pain is chronic pain. In some embodiments, the chronic pain is chronic pain associated with osteoarthritis. In some embodiments, the chronic pain is chronic pain associated with osteoarthritis of the knee. In some embodiments, the chronic pain is chronic musculoskeletal pain. In some embodiments, the chronic pain is chronic musculoskeletal pain of the lower back. In some embodiments, the pain is from an abdominal incision. In some embodiments, the pain is from an abdominoplasty. In some embodiments, the pain is from repair of an inguinal hernia. In some embodiments, the pain is from a laparotomy. In some embodiments, the pain is from a laparotomy selected from a laparotomy to repair a ventral hernia, a C-section, hysterectomy, intestinal resection, and nephrectomy. In some embodiments, the pain is from a laparotomy to repair a ventral hernia. In some embodiments, the pain is from a C-section. In some embodiments, the pain is from a hysterectomy. In some embodiments, the pain is from an intestinal resection. In some embodiments, the pain is from a nephrectomy. In some embodiments, the pain is from general surgery. In some embodiments, the pain is from obstetric and gynecological surgery. In some embodiments, the pain is from plastic surgery.

In some embodiments, 10 mL to 150 mL of the liquid pharmaceutical formulation is administered to the patient.

### EXAMPLES

These examples are provided for illustrative purposes only and not to limit the scope of the claims provided herein.

### Example 1A: Solid Pharmaceutical Composition

(E)-2-methoxy-4-((8-methylnon-6-enamido)methyl)phenyl 2-((methylamino)methyl)piperidine-1-carboxylate (Compound 1), hydrochloride salt; 15 mg, 36 mg and 60 mg free base eq/vial lyophilized drug product:

Bulk solutions were prepared with either of the following formulations:

| | Concentration | Quantity (g for |
|---|---|---|
| Ingredient | (mg/mL) | a 10 kg batch) |
| Compound 1 HCl | 12.95 | 129.5 |
| Mannitol | 20 | 200.0 |
| Citric Acid, monohydrate | 4.139 | 41.4 |
| Sodium Citrate, dihydrate | 1.559 | 15.6 |
| Water for injection | N/A | QS to 10 kg |

| | Concentration | Quantity (g for |
|---|---|---|
| Ingredient | (mg/mL) | a 10 kg batch) |
| Compound 1 HCl | 12.95 | 129.5 |
| Mannitol | 16 | 160 |
| Citric Acid, monohydrate | 3.31 | 33.1 |
| Sodium Citrate, dihydrate | 1.25 | 12.5 |
| Water for injection | N/A | QS to 10 kg |

70% of the required water was added to a glass container with a stir bar or impellor mixer. Mannitol was added to the water with mixing until dissolved. Citric acid was added to the solution with mixing until dissolved. Sodium citrate was added to the solution with mixing until dissolved. (E)-2-methoxy-4-((8-methylnon-6-enamido)methyl)phenyl 2-((methylamino)methyl)piperidine-1-carboxylate (Compound 1) was added to the solution with mixing until dissolved. The pH of the solution was measured and adjusted to 3.0 to 3.2 as necessary with 0.1 N HCl. Added remaining water to Q.S. to the appropriate weight. The solution was sterile filtered into a sterile filling bag in a Class A environment. Vials were filled aseptically to a specified volume, partially stoppered, lyophilized, and then capped.

### General Lyophilization Cycle

The vials were frozen to −50° C. Primary drying was initiated at this temperature and continued as the temperature was increased −15° C., while a vacuum of 0.133 mBar was applied to the system for approximately 38 hours. Secondary drying occurred at a higher temperature of 25° C. for approximately 12 hours, after which point the vacuum was broken and the vials were stoppered and then capped.

Solid pharmaceutical formulations of the following composition and dose were prepared by this procedure:

| | | Fill volume in vial | | | |
|---|---|---|---|---|---|
| | | 1.25 mL | 3.0 mL | 3.0 mL | 5.0 mL |
| | | mg of Compound 1 (free base) per vial | | | |
| | | 15 mg | 36 mg | 36 mg | 60 mg |
| Components | Function | weight of components | | | |
| Compound 1 | Active | 16.185 | 38.844 | 38.844 | 64.74 |
| HCl (mg) | Pharmaceutical | | | | |
| | Ingredient (API) | | | | |
| Mannitol, | Bulking | 25 | 60 | 48 | 100 |
| USP (mg) | agent | | | | |
| Citric acid, | Buffering | 5.174 | 12.418 | 9.934 | 20.70 |
| monohydrate, | agent | | | | |
| USP (mg) | | | | | |
| Sodium citrate, | Buffering | 1.949 | 4.678 | 3.742 | 7.796 |
| dihydrate, | agent | | | | |
| USP (mg) | | | | | |
| Water for | Processing aid | negligible | negligible | negligible | negligible |
| Injection, | removed during | | | | |
| USP | manufacture | | | | |

### Example 1B: Solid Pharmaceutical Composition

(E)-2-methoxy-4-((8-methylnon-6-enamido)methyl)phenyl 2-((methylamino)methyl)piperidine-1-carboxylate (Compound 1), hydrochloride salt; 2.5 and 12.5 mg/vial:

Solutions containing 20 mg/mL mannitol and various ratios of citric acid and sodium citrate to provide the specified buffer concentration at pH 3. Compound 1 HCl was added and after dissolution the solutions were each pipetted into 10 mL vials, 1.0 mL per vial. The vials were partially stoppered, lyophilized similarly to Example 1A and then capped. The following solid pharmaceutical formulations of Compound 1 were prepared:

| Example | 1B-1 | | 1B-2 | | 1B-3 | |
|---|---|---|---|---|---|---|
| Components | | | | | | |
| Compound 1 (free base) | 2.5 | mg | 12.5 | mg | 12.5 | mg |
| Mannitol, USP | 20 | mg | 20 | mg | 20 | mg |
| Citrate buffer, pH 3 | 25 | μmol | 25 | μmol | 15 | μmol |

| | | | |
|---|---|---|---|
| molar ratio of buffer to | 4.6 | 0.92 | 0.55 |
| Compound 1 | | | |
| Water for Injection, USP | negligible | negligible | negligible |

### Example 2A: Liquid Pharmaceutical Formulation

Fifty (50) mL of sterile saline, 0.9%, was taken up in a sterile syringe. A portion of the saline, 3 to 8 mL, was injected into a 15 mg vial of Compound 1 from Example 1A. The vial was swirled to reconstitute the solid within 1 minute and the contents were withdrawn via the syringe. The contents of the syringe were transferred to a sterile bowl or container to provide a total of 50 mL of a solution containing 15 mg of Compound 1.

### Example 2B: Liquid Pharmaceutical Formulation

To a 36 mg vial of Compound 1 from Example 1A, was added 5 mL of sterile saline, 0.9% via a sterile syringe. After the vial was swirled to reconstitute the solid, the liquid contents were withdrawn via the syringe. The contents of the syringe were transferred to a sterile bowl or container. An additional 115 mL of sterile saline was added to the bowl either before or after additional of the Compound 1 solution to provide a 120 mL of solution containing 0.3 mg/mL of Compound 1.

### Example 2C: Liquid Pharmaceutical Formulation

A 36 mg vial of Compound 1 from Example 1A was attached to a bag containing 120 mL of sterile saline, 0.9% via a sterile connection. Using standard procedures, a portion of the saline was transferred to the vial and then back to the bag several times to provide approximately 120 mL of a 0.3 mg/mL solution of Compound 1.

The examples and embodiments described herein are for illustrative purposes only and in some embodiments, various modifications or changes are to be included within the purview of the disclosure and scope of the appended claims.

What is claimed is:

1. A solid pharmaceutical composition comprising:
(a) about 0.25 mg to about 125 mg of (E)-2-methoxy-4-((8-methylnon-6-enamido)methyl)phenyl 2-((methylamino)methyl)piperidine-1-carboxylate, or a pharmaceutically acceptable salt thereof, and
(b) about 0.25 mg to about 75 mg of a buffering agent.

2. The solid pharmaceutical composition of claim 1, comprising about 1 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof.

3. The solid pharmaceutical composition of claim 2, comprising about 3 mg to about 75 mg of (E)-2-methoxy-4-((8-methylnon-6-enamido)methyl)phenyl 2-((methylamino)methyl)piperidine-1-carboxylate, or a pharmaceutically acceptable salt thereof.

4. The solid pharmaceutical composition of any one of claims 1-3, comprising of (E)-2-methoxy-4-((8-methylnon-6-enamido)methyl)phenyl 2-((methylamino)methyl)piperidine-1-carboxylate hydrochloride salt.

5. The solid pharmaceutical composition of any one of claims 1-4, comprising about 0.5 mg to about 45 mg of a buffering agent.

6. The solid pharmaceutical composition of any one of claims 1-5, comprising about 2 mg to about 35 mg of a buffering agent.

7. The solid pharmaceutical composition of any one of claims 1-6, wherein the buffering agent is selected from citrate, acetate, formate, glycine, maleate, tartrate, fumarate, and succinate.

8. The solid pharmaceutical composition of any one of claims 1-7, wherein the buffering agent is a citrate buffer.

9. The solid pharmaceutical composition of any one of claims 1-8, wherein the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.2 to about 10.

10. The solid pharmaceutical composition of any one of claims 1-9, wherein the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 3.

11. The solid pharmaceutical composition of any one of claims 1-10, wherein the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.5.

12. The solid pharmaceutical composition of any one of claims 1-11, further comprising a bulking agent.

13. The solid pharmaceutical composition of claim 12, comprising about 0.1 mg to about 200 mg of a bulking agent.

14. The solid pharmaceutical composition of claim 13, comprising about 1.0 mg to about 150 mg of a bulking agent.

15. The solid pharmaceutical composition of any one of claim 14, comprising about 10 mg to about 125 mg of a bulking agent.

16. The solid pharmaceutical composition of any one of claims 12-15, wherein the bulking agent is selected from mannitol, lactose, and polyvinylpyrrolidone.

17. The solid pharmaceutical composition of any one of claims 12-16, wherein the bulking agent is mannitol.

18. The solid pharmaceutical composition of any one of claims 12-16, wherein the bulking agent is lactose.

19. The solid pharmaceutical composition of any one of claims 1-18, wherein the solid pharmaceutical composition is provided in a sterile container.

20. The solid pharmaceutical composition of any one of claims 1-19, wherein the solid pharmaceutical composition is provided in a sterile container that allows for initial dilution within the container and optionally subsequent further dilution within or outside the container.

21. The solid pharmaceutical composition of claim 19 or claim 20, wherein the sterile container comprises a vial with stopper.

22. The solid pharmaceutical composition of claim 19 or claim 20, wherein the sterile container comprises a syringe cartridge.

23. The solid pharmaceutical composition of claim 19 or claim 20, wherein the sterile container comprises a syringe cartridge comprising a two component syringe that also contains a diluent.

24. The solid pharmaceutical composition of claim 19 or claim 20, wherein the sterile container comprises a kit or assembly of separate components packaged for a single use comprising one or more of the following components: diluent, container to hold a reconstituted solution of the solid pharmaceutical composition dissolved in the diluent, syringes, or devices to connect the container to other containers for transfer of the solid pharmaceutical composition, the diluent, or the reconstituted solution.

25. A solid pharmaceutical formulation comprising:
a) about 3-75 mg of (E)-2-methoxy-4-((8-methylnon-6-enamido)methyl)phenyl 2-((methylamino)methyl)piperidine-1-carboxylate hydrochloride salt;
b) about 20-150 micromoles of citrate buffer; and
c) about 10-100 mg of a bulking agent.

26. A solid pharmaceutical formulation comprising:
a) about 16 mg of (E)-2-methoxy-4-((8-methylnon-6-enamido)methyl)phenyl 2-((methylamino)methyl)piperidine-1-carboxylate hydrochloride salt;
b) about 5 mg citric acid monohydrate;
c) about 2 mg sodium citrate, dihydrate; and
d) about 25 mg of mannitol.

27. A solid pharmaceutical formulation comprising:
a) about 39 mg of (E)-2-methoxy-4-((8-methylnon-6-enamido)methyl)phenyl 2-((methylamino)methyl)piperidine-1-carboxylate hydrochloride salt;
b) about 12 mg citric acid monohydrate;
c) about 5 mg sodium citrate, dihydrate; and
d) about 60 mg of mannitol.

28. A solid pharmaceutical formulation comprising:
a) about 39 mg of (E)-2-methoxy-4-((8-methylnon-6-enamido)methyl)phenyl 2-((methylamino)methyl)piperidine-1-carboxylate hydrochloride salt;
b) about 10 mg citric acid monohydrate;
c) about 4 mg sodium citrate, dihydrate; and
d) about 48 mg of mannitol.

29. A solid pharmaceutical formulation comprising:
a) about 65 mg of (E)-2-methoxy-4-((8-methylnon-6-enamido)methyl)phenyl 2-((methylamino)methyl)piperidine-1-carboxylate hydrochloride salt;
b) about 21 mg citric acid monohydrate;
c) about 8 mg sodium citrate, dihydrate; and
d) about 100 mg of mannitol.

30. A liquid pharmaceutical formulation comprising (E)-2-methoxy-4-((8-methylnon-6-enamido)methyl)phenyl 2-((methylamino)methyl)piperidine-1-carboxylate, or a pharmaceutically acceptable salt thereof, wherein the concentration of (E)-2-methoxy-4-((8-methylnon-6-enamido)methyl)phenyl 2-((methylamino)methyl)piperidine-1-carboxylate, free base, is about 0.02 to about 2 mg/mL, the pH is 3.0 to 6.0, and the osmolarity is about 300 mOsm/kg to about 600 mOsm/kg.

31. The liquid pharmaceutical formulation of claim 30, wherein the pH is 3.4 to 5.0.

32. The liquid pharmaceutical formulation of claim 30 or claim 31, wherein the concentration of (E)-2-methoxy-4-((8-methylnon-6-enamido)methyl)phenyl 2-((methylamino)methyl)piperidine-1-carboxylate, free base, is about 0.05 mg/mL to about 1 mg/mL.

33. The liquid pharmaceutical formulation of any one of claims 30-32, wherein the concentration of (E)-2-methoxy-4-((8-methylnon-6-enamido)methyl)phenyl 2-((methylamino)methyl)piperidine-1-carboxylate, free base, is about 0.1 mg/mL to about 0.75 mg/mL.

34. The liquid pharmaceutical formulation of any one of claims 30-33, comprising (E)-2-methoxy-4-((8-methylnon-6-enamido)methyl)phenyl 2-((methylamino)methyl)piperidine-1-carboxylate hydrochloride salt.

35. The liquid pharmaceutical formulation of any one of claims 30-34, further comprising a buffering agent.

36. The liquid pharmaceutical formulation of claim 35, wherein the buffering agent is selected from citrate, acetate, formate, glycine, maleate, tartrate, fumarate, and succinate.

37. The liquid pharmaceutical formulation of claim 36, wherein the buffering agent is a citrate buffer.

38. The liquid pharmaceutical formulation of any one of claims 35-37, wherein the buffer concentration is about 0.05 mM to about 10 mM.

39. The liquid pharmaceutical formulation of any one of claims 35-38, wherein the buffer concentration is about 0.1 mM to about 5 mM.

40. The liquid pharmaceutical formulation of any one of claims 35-39, wherein the buffer concentration is about 0.2 mM to about 2 mM.

41. The liquid pharmaceutical formulation of any one of claims 30-40, wherein the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.25 to about 10.

42. The liquid pharmaceutical formulation of any one of claims 30-41, wherein the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 3.

43. The liquid pharmaceutical formulation of any one of claims 30-42, wherein the molar ratio of buffer to (E)-2-methoxy-4-((8-methylnon-6-enamido)methyl)phenyl 2-((methylamino)methyl)piperidine-1-carboxylate, or a pharmaceutically acceptable salt thereof, is about 0.5 to about 1.5.

44. The liquid pharmaceutical formulation of any one of claims 30-43, further comprising a bulking agent.

45. The liquid pharmaceutical formulation of any one of claims 30-44, further comprising about 0.005 to 0.4 weight percent of a bulking agent.

46. The liquid pharmaceutical formulation of any one of claims 30-45, further comprising about 0.01 to 0.1 weight percent of a bulking agent.

47. The liquid pharmaceutical formulation of any one of claims 44-46, wherein the bulking agent is selected from mannitol, lactose, and polyvinylpyrrolidone.

48. The liquid pharmaceutical formulation of any one of claims 44-47, wherein the bulking agent is mannitol.

49. The liquid pharmaceutical formulation of any one of claims 44-47, wherein the bulking agent is lactose.

50. A liquid pharmaceutical formulation comprising:
a) about 0.05 mg/mL to about 0.75 mg/mL of (E)-2-methoxy-4-((8-methylnon-6-enamido)methyl)phenyl 2-((methylamino)methyl)piperidine-1-carboxylate, or a pharmaceutically acceptable salt thereof,
b) about 0.1 to 1.0 mM of a buffering agent;
c) pH of 3.0 to 6.0; and
(d) osmolality of about 300 mOsm/kg to about 600 mOsm/kg.

51. The liquid pharmaceutical formulation of claim 50, comprising about 1 mg to about 90 mg of (E)-2-methoxy-4-((8-methylnon-6-enamido)methyl)phenyl 2-((methylamino)methyl)piperidine-1-carboxylate, or a pharmaceutically acceptable salt thereof.

52. The liquid pharmaceutical formulation of claim 51, comprising about 3 mg to about 75 mg of (E)-2-methoxy-4-((8-methylnon-6-enamido)methyl)phenyl 2-((methylamino)methyl)piperidine-1-carboxylate, or a pharmaceutically acceptable salt thereof.

53. The liquid pharmaceutical formulation of claim 50, comprising about 0.05 mg/mL to about 0.6 mg/mL of (E)-2-methoxy-4-((8-methylnon-6-enamido)methyl)phenyl 2-((methylamino)methyl)piperidine-1-carboxylate, or a pharmaceutically acceptable salt thereof.

54. The liquid pharmaceutical formulation of claim 51, comprising about 0.1 mg/mL to about 0.5 mg/mL of (E)-2-methoxy-4-((8-methylnon-6-enamido)methyl)phenyl 2-((methylamino)methyl)piperidine-1-carboxylate, or a pharmaceutically acceptable salt thereof.

55. The liquid pharmaceutical formulation of any one of claims 30-54, further comprising a local anesthetic.

56. The liquid pharmaceutical formulation of any one of claims 30-55, further comprising a local anesthetic, wherein the local anesthetic is selected from bupivacaine, levobupivacaine, tetracaine, and ropivacaine.

57. A liquid pharmaceutical formulation comprising:
a) about 0.15 to 0.75 mg/mL of (E)-2-methoxy-4-((8-methylnon-6-enamido)methyl)phenyl 2-((methylamino)methyl)piperidine-1-carboxylate hydrochloride salt;
b) about 0.25 to 1.25 mM of a citrate buffer at pH 3.4 to 5.0; and
(c) osmolality of about 300 mOsm/kg to about 600 mOsm/kg.

58. A liquid pharmaceutical formulation comprising:
a) about 0.15 to 0.75 mg/mL of (E)-2-methoxy-4-((8-methylnon-6-enamido)methyl)phenyl 2-((methylamino)methyl)piperidine-1-carboxylate hydrochloride salt;
b) about 0.25 to 1.25 mM of a citrate buffer at pH 3.4 to 5.0;
c) about 0.005 to 0.4 weight percent of a bulking agent; and
d) osmolality of about 300 mOsm/kg to about 600 mOsm/kg.

59. A liquid pharmaceutical formulation comprising:
a) about 0.125 mg/mL of (E)-2-methoxy-4-((8-methylnon-6-enamido)methyl)phenyl 2-((methylamino)methyl)piperidine-1-carboxylate free base;
b) about 0.26 mM of a citrate buffer at pH 3.4 to 5.0;
c) about 0.2 mg/mL of mannitol; and
d) osmolality of about 300 mOsm/kg to about 600 mOsm/kg.

60. A liquid pharmaceutical formulation comprising:
a) about 0.3 mg/mL of (E)-2-methoxy-4-((8-methylnon-6-enamido)methyl)phenyl 2-((methylamino)methyl)piperidine-1-carboxylate free base;
b) about 0.63 mM of a citrate buffer at pH 3.4 to 5.0;
c) about 0.5 mg/mL of mannitol; and
d) osmolality of about 300 mOsm/kg to about 600 mOsm/kg.

61. A liquid pharmaceutical formulation comprising:
a) about 0.3 mg/mL of (E)-2-methoxy-4-((8-methylnon-6-enamido)methyl)phenyl 2-((methylamino)methyl)piperidine-1-carboxylate free base;
b) about 0.5 mM of a citrate buffer at pH 3.4 to 5.0;
c) about 0.4 mg/mL of mannitol; and
d) osmolality of about 300 mOsm/kg to about 600 mOsm/kg.

62. A liquid pharmaceutical formulation comprising:
a) about 0.5 mg/mL of (E)-2-methoxy-4-((8-methylnon-6-enamido)methyl)phenyl 2-((methylamino)methyl)piperidine-1-carboxylate free base;
b) about 1.05 mM of a citrate buffer at pH 3.4 to 5.0;
c) about 0.8 mg/mL of mannitol; and
d) osmolality of about 300 mOsm/kg to about 600 mOsm/kg.

63. A method of preparing a liquid pharmaceutical formulation, comprising dissolving a solid pharmaceutical composition of any one of claims 1-29 in a diluent.

64. A method of preparing a liquid pharmaceutical formulation, comprising dissolving a solid pharmaceutical composition of any one of claims 1-29 in a first portion of a diluent in a first container, and then transferring to a second container for dilution with a second portion of a diluent to a final volume and concentration.

65. The method of claim 64, wherein the first container is attached in a sterile manner to the second container by a connection device and the first portion of diluent is used to flush the liquid pharmaceutical formulation into the second container.

66. A method of preparing a liquid pharmaceutical formulation, comprising a two compartment syringe containing a solid pharmaceutical composition of any one of claims 1-29 in a first compartment and a diluent in a second compartment, wherein the solid pharmaceutical composition in the first compartment and the diluent in the second compartment are combined to allow dissolution of the solid pharmaceutical composition in the diluent directly in the syringe.

67. The method of any one of claims 63-66, wherein the diluent is a sterile solution.

68. The liquid pharmaceutical formulation of any one of claims 63-67, wherein the diluent is a saline solution.

69. The liquid pharmaceutical formulation of any one of claims 63-67, wherein the diluent is a Lactated Ringer's solution.

70. A method of treating or preventing pain in a subject in need thereof, comprising parenterally administering to the subject a liquid pharmaceutical formulation of any one of claims 30-69.

71. The method of claim 70, wherein the pain is post-surgical pain, post amputation pain, chronic post-surgical pain, and pain associated with acute traumatic injury.

72. The method of claim 71, wherein the pain is postsurgical pain.

73. The method of claim 72, wherein the postsurgical pain is pain from a laparotomy, hernia repair, thoracotomy, thoraco-abdominal incision, flank incision, total hip replacement, total knee replacement, ACL reconstruction, rotator cuff repair, bunionectomy, laparoscopy, dental extraction, or open reduction internal fixation of fractures.

74. The method of claim 71, wherein the pain is pain associated with acute traumatic injury.

75. The method of claim 74, wherein the pain associated with acute traumatic injury is pain from a long bone, short bone, flat bone, or irregular bone fracture.

76. The method of claim 74, wherein the pain associated with acute traumatic injury pain is pain from a hip or rib fracture.

77. The method of claim 76, wherein the pain is chronic post-surgical pain.

78. The method of claim 77, wherein the chronic post-surgical pain is pain after mastectomy or lumpectomy.

79. The method of claim 77, wherein the chronic post-surgical pain is pain after thoracotomy.

80. The method of claim 77, wherein the chronic post-surgical pain is pain after amputation.

81. The method of claim 77, wherein the chronic post-surgical pain is pain after hernia repair.

82. The method of claim 70, wherein the pain is chronic pain.

83. The method of claim 82, wherein the chronic pain is chronic pain associated with osteoarthritis.

84. The method of claim 82, wherein the chronic pain is chronic pain associated with osteoarthritis of the knee.

85. The method of claim 82, wherein the chronic pain is chronic musculoskeletal pain.

86. The method of claim 85, wherein the chronic pain is chronic musculoskeletal pain of the lower back.

87. The method of any one of claims 70-86, wherein 10 mL to 150 mL of the liquid pharmaceutical formulation is administered to the patient.
